(19) United States
(12) Patent Application Publication
(10) Pub. No.: US 2023/0374448 A1
(43) Pub. Date: Nov. 23, 2023

(54) Hypoimmunogenic Cells and Methods and Compositions for Their Production

(71) Applicant: RXCELL INC., Novato, CA (US)

(72) Inventors: Xianmin ZENG, Novato, CA (US); Mahendra RAO, Timonium, MD (US)

(21) Appl. No.: 17/913,535

(22) PCT Filed: Apr. 6, 2021
(86) PCT No.: PCT/US2021/025867

§ 371 Date: Sep. 22, 2022

Related U.S. Application Data
(60) Provisional application No. 63/005,651, filed on Apr. 6, 2020.

(51) Int. Cl. C12N 5/0793; C12N 15/90; C12N 5/00; C07K 14/54; C07K 14/52

(52) U.S. Cl. CPC C12N 5/0619; C12N 5/062; C12N 15/907; C12N 5/0006; C07K 14/5428; C07K 14/521; C12N 2506/45; C12N 2509/00; C12N 2501/105; C12N 2501/41; C12N 2501/119

(57) ABSTRACT

Hypoimmunogenic cell lines and methods and compositions for their production are provided.

This patent application claims the benefit of priority from U.S. Provisional Application Ser. No. 63/005,651, filed Apr. 6, 2020.

### FIELD

The present invention relates to hypoimmunogenic cell lines and methods and compositions for their production.

### BACKGROUND

The immune system plays an important role in maintaining the integrity of the organism. It recognizes self from non-self and mounts a defense against bacterial, viral and other microorganisms. The immune system is comprised of cells in the circulation as well as resident immune cells present in most tissues and organs. These resident immune cells have different names such as microglia in the central nervous system (CNS).

The origin of resident microglia was controversial until definitive studies showed that they are derived from yolk sac primitive hematopoietic precursors. The definitive proof for a mesodermal origin of microglia was achieved through a genetic study that showed that mice lacking the crucial transcription factor for myeloid cells, PU.1, are devoid of microglia (McKercher et al. EMBO J (1996) 15(20):5647-58. 22; Beers et al. Proc Natl Acad Sci USA (2006) 103(43):16021-6).

The immune system recognizes foreign antigens via two major arms of the immune system (Chaplin et al. J Allergy Clin Immunol. 2010 February; 125(2 Suppl 2):S3-23). The mechanisms permitting recognition of microbial, toxic, or allergenic structures can be broken down into two general categories referred to as innate and adaptive immune pathways.

Innate immune pathways are hard-wired responses that are encoded by genes in the host's germ line and that recognize molecular patterns shared both by many microbes and toxins that are not present in the mammalian host. The innate response also includes soluble proteins and bioactive small molecules that are either constitutively present in biological fluids (such as complement proteins, defensins, and ficolins (Hiemstra, P. S. Exp Lung Res. 2007; 33:537-542; Holmskov et al. Annu Rev Immunol. 2003; 21:547-578) or that are released from cells as they are activated (including cytokines that regulate the function of other cells, chemokines that attract inflammatory leukocytes, lipid mediators of inflammation, reactive free radical species, and bioactive amines and enzymes that also contribute to tissue inflammation). The first set of responses constitutes the innate immune response because the recognition molecules used by the innate system are expressed broadly on a large number of cells. This system is poised to act rapidly after an invading pathogen or toxin is encountered and thus constitutes the initial host response.

The second set of immune responses constitutes the adaptive immune system, also referred as the acquired immune system. Unlike the innate immune system, the acquired immune system is highly specific to a particular pathogen. Adaptive immune pathway responses are encoded by gene elements that somatically rearrange to assemble antigen-binding molecules with exquisite specificity for individual unique foreign structures. The human leukocyte antigen (HLA) system or complex is a gene complex encoding the major histocompatibility complex (MHC) proteins in humans. These cell-surface proteins are responsible for the regulation of the immune system in humans.

The innate and adaptive immune systems are often described as contrasting, separate arms of the host response; however, they usually act together, with the innate response representing the first line of host defense, and with the adaptive response becoming prominent after several days, as antigen-specific T and B cells have undergone clonal expansion. Components of the innate system contribute to activation of the antigen-specific cells. Additionally, the antigen-specific cells amplify their responses by recruiting innate effector mechanisms to bring about the complete control of invading microbes. Thus, while the innate and adaptive immune responses are fundamentally different in their mechanisms of action, synergy between them is essential for an intact, fully effective immune response.

Other important components of the immune system are derived from the hematopoietic stem cells. Multipotent hematopoietic stem cells differentiate in bone marrow into common lymphoid or common myeloid progenitor cells. Lymphoid stem cells give rise to B cell, T cell, and NK cell lineages. Myeloid stem cells give rise to a second level of lineage specific colony form unit (CFU) cells that go on to produce neutrophils, monocytes, eosinophils, basophils, mast cells, megakaryocytes, and erythrocytes. Monocytes differentiate further into macrophages in peripheral tissue compartments. Dendritic cells (DC) appear to develop primarily from a DC precursor that is distinguished by its expression of the Flt3 receptor. This precursor can derive from either lymphoid or myeloid stem cells and gives rise to both classical DC and plasmacytoid DC. Classical DC can also derive from differentiation of monocytoid precursor cells.

T cells are also an important arm of the immune system. T cells have evolved an elegant mechanism that recognizes foreign antigens together with self-antigens as a molecular complex. This requirement that T cells recognize both self-structures and foreign antigens makes the need for these cells to maintain self-tolerance particularly important.

A major role of the T cell arm of the immune response is to identify and destroy infected cells. T cells can also recognize peptide fragments of antigens that have been taken up by APC through the process of phagocytosis or pinocytosis. The way the immune system has evolved to permit T cells to recognize infected host cells is to require that the T cell recognize both a self-component and a microbial structure. The elegant solution to the problem of recognizing both a self-structure and a microbial determinant is the family of MHC molecules. MHC molecules (also called the human leukocyte-associated [HLA] antigens) are cell surface glycoproteins that bind peptide fragments of proteins that either have been synthesized within the cell (class I MHC molecules) or that have been ingested by the cell and proteolytically processed (class II MHC molecules).

There are three major HLA class I molecules, designated HLA-A, HLA-B, and HLA-C, each encoded by a distinct gene. The class I HLA molecules are cell surface heterodimers consisting of a polymorphic transmembrane 44-kd α-chain (also designated the class I heavy chain) associated with the 12-kd non-polymorphic β₂-microglobulin (β₂m) protein. The α-chain determines whether the class I molecule is an HLA-A, HLA-B, or HLA-C molecule. The HLA-A, HLA-B, and HLA-C α-chain genes are encoded within the MHC on chromosome 6, and the β₂-microglobulin gene is encoded on chromosome 15. The TCR contacts both the antigenic peptide and the flanking α-helices. The TCR has no measurable affinity for the antigenic peptide alone, and very low affinity for MHC molecules containing other peptides. It has been recognized that T cells only recognize their specific antigen when it was presented in association with a specific self-MHC molecule (Zinkernagel, R. M. and Doherty, P. C. (1977) Major Transplantation Antigens, Viruses, and Specificity of Surveillance T Cells. In: Stutman O. (eds) Contemporary Topics in Immunobiology. Contemporary Topics in Immunobiology, vol 7. Springer, Boston, MA).

β₂-microglobulin gene (B2M), which is encoded on chromosome 15, is required for appropriate expression of the Class I MHC complex. In its absence the class I HLA complex does not express or function appropriately.

Like the class I molecules, class II HLA molecules consist of two polypeptide chains, but in this case, both are MHC-encoded transmembrane proteins and are designated α and β. There are three major class II proteins designated HLA-DR, HLA-DQ, and HLA-DP. Molecules encoded in this region were initially defined serologically and using cellular immune assays, and consequently their nomenclature does not always reflect the underlying genes encoding the molecules. This is particularly true for HLA-DR, where the genes in the HLA-DR sub-region encode 1 minimally polymorphic (1 common and 2 very rare alleles) α chain (designated DRA) and 2 polymorphic β chains (designated DRB1 and DRB3).

Endogenous proteins are digested by the immunoproteasome to small peptide fragments optimal for loading into Class I molecules. Peptides are transferred from the immunoproteasome to the endoplasmic reticulum via the TAP transporter. With the help of tapasin, calreticulin and the chaperon Erp57, they are loaded into a class I heavy chain that associates with a β2m subunit via facilitation by chaperon protein calnexin prior to transport to the cell surface wherein it can be recognized by CD8+ T cells.

Recently it has also become clear that there is a class of T cells that recognizes antigens presented by molecules that are not classical HLA class I or class II antigens. One of these classes of T cells uses an antigen receptor composed of α and β chains and recognizes lipid antigens that are presented bound to CD1 molecules. CD1 molecules are structurally related to class I HLA molecules, being transmembrane proteins with 3 extracellular domains and associating with β₂-microglobulin. There are 5 human CD1 isoforms designated CD1a-CD1e, encoded by linked genes that are not associated with the MHC.

Additional innate immune effectors critical for effective host defense include phagocytic cells including neutrophils, macrophages, and monocytes and natural killer (NK) cells.

Since the immune system employs many potent effector mechanisms that have the ability to destroy a broad range of microbial cells and to clear a broad range of both toxic and allergenic substances, it is critical that the immune response be able to avoid unleashing these destructive mechanisms against the mammalian host's own tissues. The ability of the immune response to avoid damaging self-tissues is referred to as self-tolerance.

Controlling this same immune response is also important in preventing rejections of tissue, organ and bone marrow transplants between two unrelated individuals. However, while bypassing the immune system may allow for cell, tissue and organ transplants between unrelated individuals, such a bypass must not prevent the immune system from performing its other important functions.

A variety of techniques have evolved to try to modulate the immune system to allow for organ transplants including autologous or syngeneic transplant, transplant in utero, transplant to an immune privileged site, co-transplant of immune modulatory cells, localized immunosuppression, CTL blockade, anti-TCR therapy, ABO tolerization and other antigen tolerization, T-regs to induce tolerization, mixed chimerism with accompanied bone marrow transplant, generation of DC cells, thymic rejuvenation and HLA matching with immune suppression.

Several approaches for generating universal cells to prevent immune rejection have also been described.

A first approach involved use of modern gene editing technologies to knock out expression of all Class I and Class II genes (Lanza, Russell and Nagy 2019). Modifications of this strategy with largely the same goals have been used by a variety of other investigators including using other variants of HLA-G, using HLA-E or knocking out each HLA-class individually or assuming Class II knock out will not be necessary. In general, while these strategies substantially inhibited adaptive immunity there was only a modest reduction or an increase in the innate immune response leading to chronic rejection.

Recognizing the limitations of the Class I and Class II knockouts, it has been proposed that inhibiting all the arms of the immune response by selecting 1-2 molecules that inhibit each pathway as an alternate approach (Lanza, Russell and Nagy 2019). This approach has several theoretical advantages to the KO approach in that the molecules used are used in specialized cases where HLA mismatches exist, and a tissue or cell graft is tolerated. Although this method has many advantages, there are some very practical disadvantages in that as many as eight genes were used and the levels of expression required were high with multiple copies of the transgene being required.

Using a hybrid approach, Cowan et al. have shown that adding three immunomodulatory genes to a Class I and Class II double KO while simultaneously co-expressing three genes may be sufficient. However, the three genes they used modulate the checkpoint pathway (CD47, PDL1 and HLA-G) cause substantial, but not complete, reduction in immune activity (Cowan et al. Nat. Biotechnol. 2019 37:252-258).

Using a similar hybrid strategy, Deuse et al. showed that a single gene may be sufficient. Using CD47, they showed inhibition of NK cell activity and that appeared sufficient to enable long-term graft survival. Further, both mouse and human induced pluripotent stem cells (iPSC) were shown to lose their immunogenicity when class I and II genes are inactivated and CD47, acting as a checkpoint inhibitor, was over-expressed. However, deleting HLA class I and II expression on hESCs and their derivatives (endothelial cells and vascular smooth muscle cells) and knocking in HLA-G, CD47 and PD-L1 in vitro and in mice studies showed T-cell responses to only be blunted, not abolished, thus raising questions about the Deuce report and suggesting that levels of expression of CD47 may be critical.

Engineered HLA class Ia-negative hESCs with knock in of HLA-E have also been produced. These hESCs and their differentiated derivatives (CD45⁺ hematopoietic cells) escaped allogeneic CD8⁺ T cell responses and were resistant to cytolysis by CD94/NKG2A− positive NK cells, both in vitro and in mice. However, issues have arisen with translating this approach to humans since only around 50% of human NK cells express CD94/NKG2A, the receptor which binds to HLA-E to inhibit NK cells. Thus, HLA class Ia-negative, HLA-E-expressing allogeneic cells may still be eliminated by CD94/NKG2A− negative NK cells.

iPSCs and their derivatives (endothelial cells and cardiomyocytes) engineered to be HLA class I and II deficient and to overexpress CD47 were shown in vitro and in vivo to be protected from NK cell responses and immune rejection. Importantly, derivatives were able to survive long-term without immune suppression in mismatched, immune-competent, allogeneic, humanized mice. However, a fifth of endothelial cell allografts failed without explanation.

Thus, there is a need for hypoimmunogenic cells and methods and compositions for their production which do not invoke an immune response.

### SUMMARY

An aspect of the present invention relates to a cell modified to be hypoimmunogenic which does not express Class I epitopes or Class II epitopes and which overexpresses IL-10 factor or MIF factor.

In one nonlimiting embodiment, the cell does not express both Class I and Class II epitopes.

In one nonlimiting embodiment, the cell overexpresses IL-10 factor and MIF factor.

Another aspect of the present invention relates to a cell modified to be hypoimmunogenic which overexpresses IL-10 factor and/or MIF factor and expresses additional factors which mimic the loss of Class I and/or Class II activity.

Another aspect of the present invention relates to a method for production of a cell which does not activate innate or adaptive immune responses. In one nonlimiting embodiment, the cell is modified to not express Class I and/or Class II epitopes and to overexpress IL-10 factor and/or MIF factor. In one nonlimiting embodiment, the cell is modified to overexpress IL-10 factor and/or MIF factor and to express additional factors which mimic the loss of Class I and/or Class II activity.

Yet another aspect of the present invention relates to a nucleic acid construct for insertion or integration into a cell, thus modifying the cell to be hypoimmunogenic. In one nonlimiting embodiment, the nucleic acid construct comprises an IL-10 gene or a MIF gene or both an IL-10 gene and a MIF gene driven by a promoter such as the CAG promoter inserted into a safe harbor site such as on Chr. 13.

### BRIEF DESCRIPTION OF THE FIGURES

FIG. 1 provide a nonlimiting example of a master design of generating hypoimmunogenic cell lines in accordance with the present invention by knocking out Class I or Class II epitopes or both and I and Class II epitopes, and overexpressing immunomodulatory factors IL-10 or MIF or both in safe harbors such as the CLYBL on Chr. 13 or AAVS1 on Chr. 19.

FIGS. 2A through 2C provide an example of lines with Class I KO of B2M, RCL-B-1 iPSC line, using an iPSC line (NCL2-GFP) as the parental line. FIG. 2A shows gene and guide RNA information. FIG. 2B shows genotype analysis. FIG. 2C shows karyotype analysis.

FIGS. 3A and 3B provide an example of lines with Class I (B2M) and Class II (CIITA) double KO, RCL-BC-1 iPSC line, using an iPSC line (NCL2-GFP) as the parental line. FIG. 3A shows gene and guide RNA information. FIG. 3B shows karyotype analysis. Double KO of the B2M and CIITA genes in both alleles was confirmed by DNA sequencing.

FIGS. 4A through 4C show an example of an HLA-KO (B2M/CIITA double KO) hypoimmunogenic iPSC line overexpressing IL-10 and MIF in a safe harbor site (AAVS1 on Chr. 19). FIG. 4A shows a targeting vector expressing IL-10 and MIF at the AAVS1 site on Chr. 19 used to generate the line. FIG. 4B shows DNA sequencing verification of the correct targeting. FIG. 4C shows PCR verification of the correct targeting.

FIG. 5 is a graph showing results of an in vitro immunogenicity assay wherein NK cell killing was measured by LDH toxicity. As shown, NK cell responses were diminished in hypoimmunogenic cells expressing IL-10 or MIF or both by either knocking in at safe harbor sites or by random integration such as transposon. The IL-10 and MIF expression lines (HLA-KO+IL10-MIF lines) elicited less NK cell killing than the control and an HLA-KO cell not expressing IL-10 and MIF. The bar graph represents the % of NK cytotoxicity against neural progenitors derived from each line. Different effector/target (E/T) ratios are used in the experiment and the figure showed the result of a 1:3 E/T e=ratio.

### DETAILED DESCRIPTION

Disclosed herein are cell lines modified to be hypoimmunogenic thereby not activating innate or adaptive immune responses and compositions and methods for production of these cell lines.

In one nonlimiting embodiment of these hypoimmunogenic cell lines, expression of Class I or Class II epitopes or both is eliminated while expression of IL-10 factor or MIF factor or both is increased. In another nonlimiting embodiment of these hypoimmunogenic cell lines, expression of IL-10 factor or MIF factor or both is increased and additional factors which mimic the loss of Class I and/or Class II activity are added.

In one nonlimiting embodiment of the present invention, the hypoimmunogenic cell line is an iPSC or an immortalized line. However, as will be understood by the skilled artisan upon reading this disclosure, any proliferating cell which undergoes sufficient cell divisions to allow insertion and selection of a cell with desired properties of having expression of Class I or Class II epitopes eliminated while expression of IL-10 factor or MIF factor or both is increased can be used. Nonlimiting examples of such cells include mesenchymal stem cells, neural stem cells and hematopoietic stem cells.

Interleukin-10 (IL-10) is a key immunosuppressive cytokine that is produced by a wide range of leukocytes as well as non-hematopoietic cells (Shouval et al., 2014). IL-10 mediates its anti-inflammatory effects through IL-10R-dependent signals emanating from the cell surface. The IL-10R is a hetero-tetramer that consists of two subunits of IL-10Rα and two subunits of IL-10Rβ (Moore et al., 2001). While the IL-10Rα subunit is unique to IL-10 signaling, the IL-10Rβ subunit is shared by other cytokine receptors, including IL-22, IL-26 and IFN-λ (Moore et al., 2001). IL-10 downstream signaling through the IL-10R inhibits the induction of pro-inflammatory cytokines by blocking NF-κB-dependent signals (Saraiva and O'Garra, 2010). Loss of this pathway is known to cause proinflammatory auto immune disease such as ulcerative colitis. More recently, it has been shown that this action is because of the loss of modulation of the innate immune system and by extension likely due to its actions on macrophages, phagocytes and NK cells.

The pro-inflammatory cytokine macrophage migration inhibitory factor (MIF) stimulates tumor cell proliferation, migration and metastasis, promotes tumor angiogenesis, suppresses p53-mediated apoptosis and inhibits anti-tumor immunity by largely unknown mechanisms. While overexpression of MIF in ovarian cancer has been correlated with malignancy and the presence of ascites, functionally MIF contributes to the immune escape of OvCA by transcriptionally downregulating NKG2D in vitro and in vivo, thus impairing natural killer (NK) cell cytotoxicity towards tumor cells (O'Reilly et al. Med Res Rev. 2016 May; 36(3):440-60). Further relevance of NKG2D-mediated reactions against ovarian carcinomas was recently demonstrated in adoptive transfer of transgenic T cells expressing a chimeric NKG2D-CD3 ξ (receptor which induced rejection even of late-stage tumors independent of the presence of known tumor antigens since NKG2D ligands providing both T cell receptor activation and co-stimulation (Bloom et al. Expert Opin Ther Targets. 2016 December; 20(12):1463-1475).

MIF also impairs anti-tumor immunity by inhibiting CTL and NK cell responses (Krockenberger et al. J Immunol. 2008 Jun. 1; 180(11): 7338-7348), an effect proposed to be caused by MIF-induced T cell activation followed by activation-induced cell death (Yan et al. Cytokine. 2006 Feb. 21; 33(4): 188-198). However, activation of NK cells by MIF has not been observed this indicating that the MIF-mediated inhibition of NK cells is effected via a different mechanism (Rosen et al. J Immunol 2004; 173:2470-2478). In mice, NK cell activation and T cell co-stimulation are mediated via two different isoforms of NKG2D that recruit specifically either DAP10 or DAP12. In humans, however, the “NKG2Dₛₕₒᵣₜ”-DAP12 pathway does not appear to exist (Unruh et al. Am J Kidney Dis. 2019 March; 73(3): 429-431).

In addition, MIF is a pleiotropic cytokine shown to have a protective role in the heart during ischemia-reperfusion injury after cardiac surgery. Multiple cell types store MIF in intracellular pools and release it in response to stress and other mediators. After liberation from the cell, MIF may bind to various receptors, including CXCR2, CXCR4, and CD74. When binding CXCR2 or CXCR4, MIF promotes inflammation. MIF binding to CD74 appears to have beneficial effects in the heart during ischemia-reperfusion injury by an antioxidant mechanism through the CD74/CD44/AMP-activated protein kinase. Downstream mechanisms that underlie MIF-induced renoprotection in the setting of ischemic injury are still under investigation. However, some studies have shown that AMPK activation (or pre-activation, mimicking ischemic preconditioning) has beneficial effects in dampening kidney injury in rodent ischemia-reperfusion injury models and in liver injury (Miller et al. Nature. 2008 Jan. 31; 451(7178):578-82).

MIF has not usually been considered in cell transplant therapy because of its proinflammatory as well as anti-inflammatory activity.

However, in the present invention involving HLA null cells, it is expected that MIF will play a largely inhibitory role. This inhibition will be of the innate immune pathway with little or no activation of the adaptive immune pathway as the other co-stimulators of the adaptive pathway are absent. While proinflammatory in most circumstances, in specific cases MIF is potently anti-inflammatory allowing cancers to escape immune surveillance. Accordingly, in the present invention, MIF overexpression is used to enable HLA null cells to escape immune surveillance while taking advantage of the protective role of MIF in enhancing cell survival to trauma and injury.

Using this hybrid approach of the present invention, Class I and/or Class II knockout cell lines such as, but not limited to, B2M and CTIIA cell lines were created which express of IL-10 and/or MIF. Such cells were created using either knocked in safe harbor sites (AAVS1 and CYBYL), transfection by transposon or lentiviral trasnfection. Unlike other hybrid approaches, further modulation of the adaptive immune system via checkpoint inhibitors having a specific mechanism of action such as CD200, PDL1 and CD47 is not required. Instead, only IL-10 and/or MIF molecules specific to the innate immune pathway which are well recognized in producing tolerance via instructing cells to change their phenotype to a T-reg or regulatory macrophage phenotype need to be overexpressed. These molecules are normally expressed by resident cells, in particular resident macrophages and microglia of the CNS. Further, IL-10 and MIF are often overexpressed in glioblastomas, thus playing a key role in regulating tumor growth and escape from the immune system.

In one nonlimiting embodiment of the present invention, knocking out the Class I locus is achieved by knocking out B2M gene which is a component of the Class I molecules. In another nonlimiting embodiment, knocking out the Class I locus is achieved by knocking out genes (HLA-A, HLA-B and HLA-C) individually. In another nonlimiting embodiment, inhibiting expression of Class I antigens is achieved by knocking out processing enzymes such as, but not limited to, tapasin, which prevent surface expression of the Class I antigens.

In one nonlimiting embodiment, knocking out the Class II locus is achieved by knocking out a master transcription factor such as the RFANX or CIITA gene that is required for Class II gene expression.

In one nonlimiting embodiment of the hypoimmunogenic cell lines of the present invention, expression of Class I or Class II epitopes is eliminated while expression of IL-10 factor or MIF factor is increased. In one nonlimiting embodiment of the hypoimmunogenic cell lines of the present invention, Class I and Class II epitopes are eliminated while expression of IL-10 factor or MIF factor is increased. In one nonlimiting embodiment of the hypoimmunogenic cell lines of the present invention, Class I or Class II epitopes are eliminated while expression of IL-10 and MIF are increased.

In yet another nonlimiting embodiment of the hypoimmunogenic cell lines of the present invention, Class I and Class II epitopes are eliminated while expression of IL-10 and MIF are increased. It is expected that this combination of modifications to the cells may be most useful in production of a cell which does not activate innate or adaptive immune responses since cells may autoregulate the levels of the receptor, and because of the pleiotrophic nature of these molecules, lower levels of each cytokine can be used. Synergistic activity of this nonlimiting embodiment is also expected as these molecules act via distinct receptors, activate different cytokines, and modulate other arms of the immune system differently.

In addition, additional factors known to enhance activity of IL-10 and MIF such as, but not limited to IL-4 and IL-35 can be added to the cells to further enhance their activity and therapeutic utility.

Various methods for producing these hypoimmunogenic cell lines can be used. Nonlimiting examples include safe harbor insertion, transfection by transposon, random integration by lentivirus, also referred to as lenti viral transfection, and transposon technology including but not limited to Piggy-Bac, sleeping beauty, Tol-2, and other technologies.

In another nonlimiting embodiment of the hypoimmunogenic cell lines of the present invention, expression of IL-10 factor or MIF factor or both is increased and additional factors which mimic the loss of Class I and/or Class II activity are added. Nonlimiting examples of such factors which can be added to mimic the loss of Class I and/or Class II activity include antisense oligonucleotides or siRNA or modifications for translation that reduce the expression of HLA antigens on the cell's surface and factors that block T and B cell function or alter inflammatory response such as IL-35, CD200 and PD-L1. Methods of insertion of these additional factors include, but are not limited to, electroporation, nucleofection, lipofection and lentivirus or other viral vectors.

The present invention also provides nucleic acid constructs for insertion or integration into a cell line, thus modifying the cell line to be hypoimmunogenic. In one nonlimiting embodiment, the nucleic acid construct comprises an IL-10 gene or a MIF gene or both an IL-10 gene and a MIF gene. Expression of the gene product can be driven by an endogenous promoter when the insertion is in frame with an endogenous gene, or by an exogenous promoter such as CAG or EF-alpha or any other well-known exogenously supplied promoter with the limitation that this be expressed in the cell product to which it is being inserted or integrated. The construct may be inserted into a known safe harbor locus such as the AAVS1 site on Chr. 19 or a Chr. 13 site such as CLYBL. In accordance with the present invention, the nucleic acid construct is inserted or integrated into a cell line in with Class I epitopes or Class II epitopes or Class II epitopes and Class II epitopes have been knocked out.

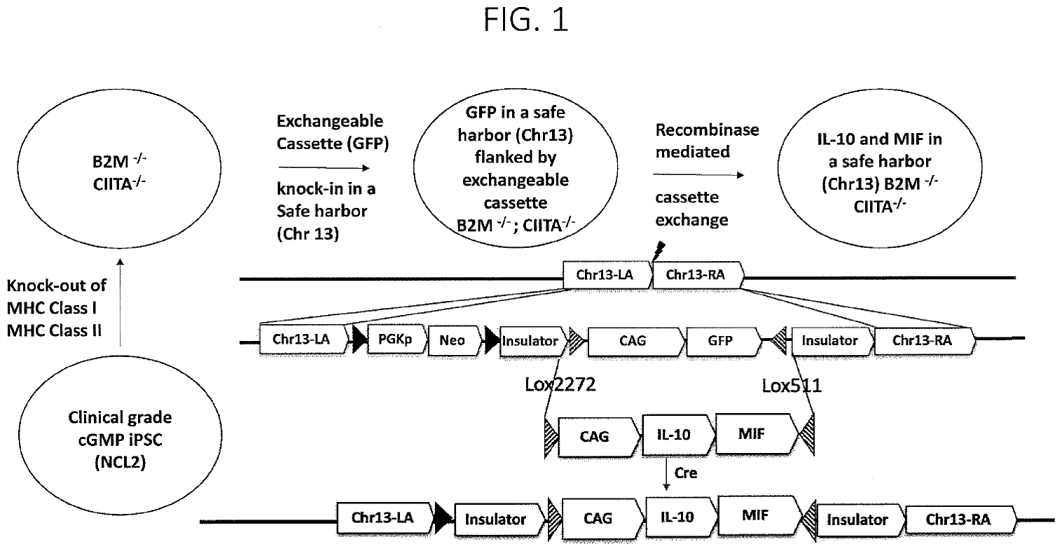

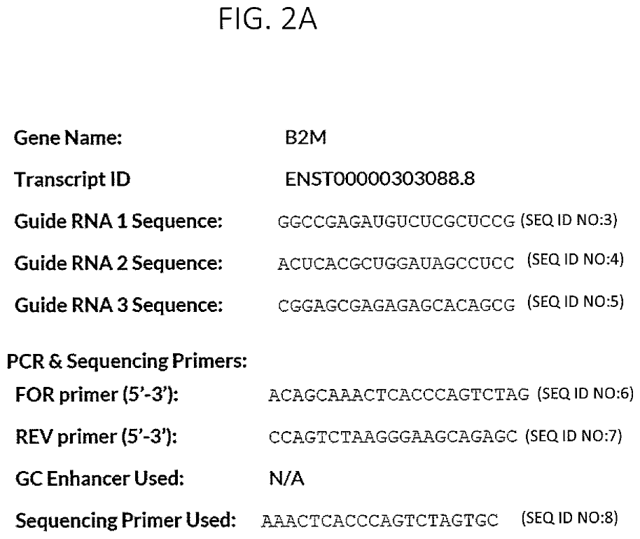

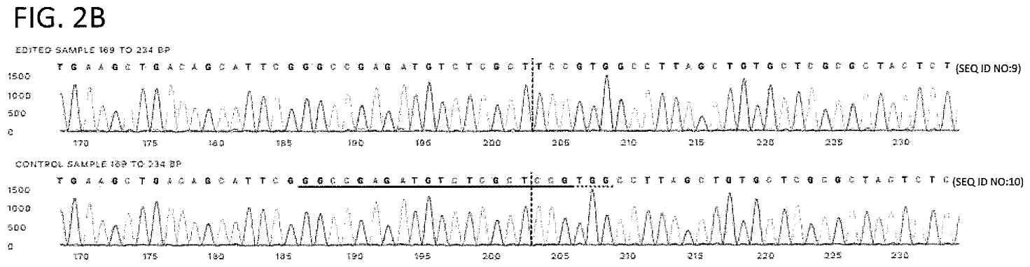

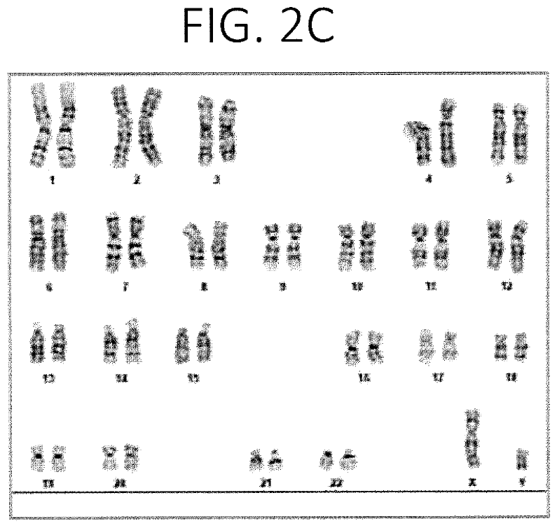

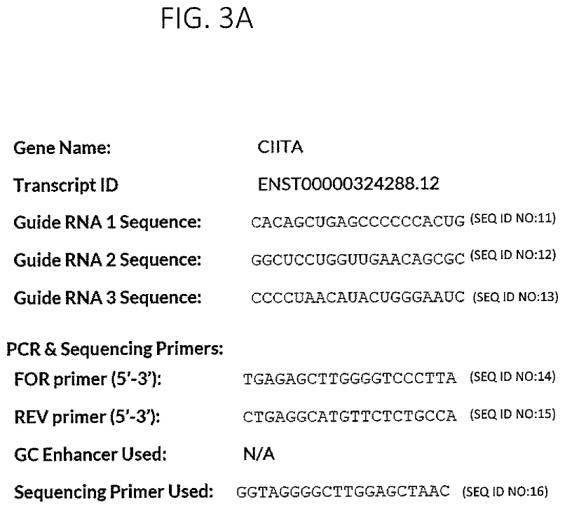

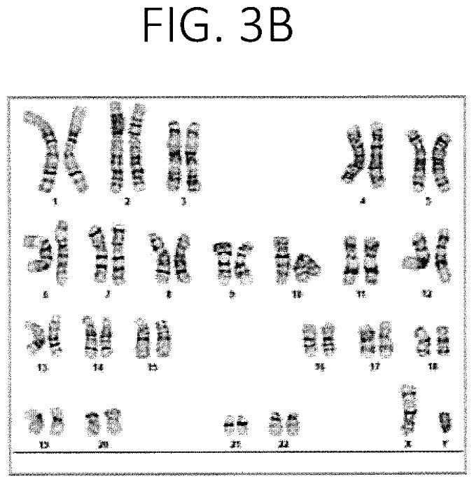

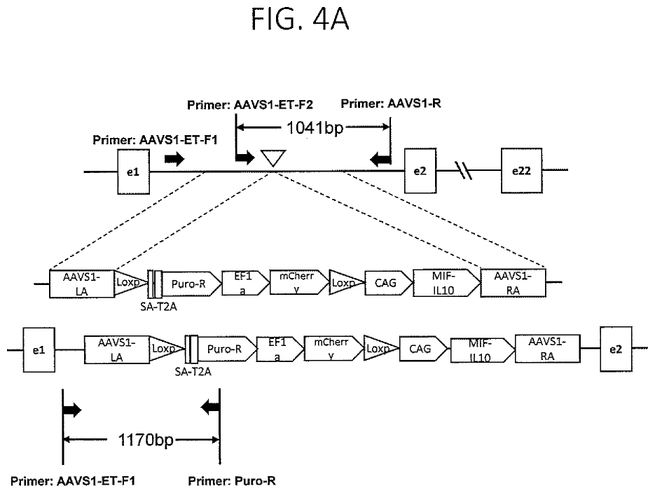

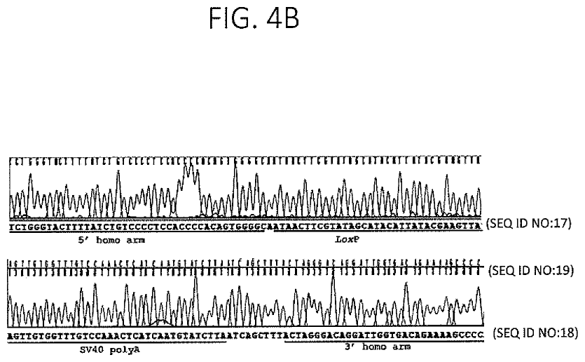

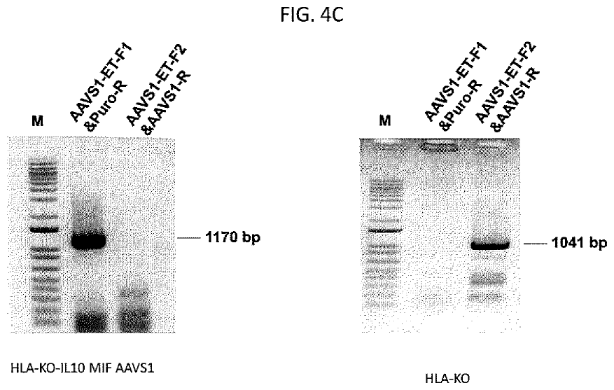

Various nonlimiting examples of hypoimmunogenic cells lines were produced in accordance with the present invention. A material design for creation of a hypoimmunogenic cell line in accordance with the present invention is set forth in FIG. 1. A nonlimiting example of a hypoimmunogenic line with Class I knock-out gene is set forth in FIG. 2 and a nonlimiting example of a hypoimmunogenic line with Class I and II double knock-out set forth in FIG. 3. Additional nonlimiting examples of cell lines created in accordance with the present invention include NCL2-GFP, an iPSC line with GFP knocked in a safe harbor site CLYBL on Chr. 13 produced using NCL2, a cGMP clinical grade iPSC line as the parental line; RCL-B-1, an HLA Class I (B2M) knock-out iPSC line produced using NCL2-GFP as the parental line; RCL-BC-1, an HLA Class I (B2M) and Class II (CIITA) double knock-out iPSC line produced using NCL2-GFP as the parental line; RCL-BC-1-IL, an iPSC line generated by knocking-out both HLA Class I (B2M) and Class II (CIITA) genes, and knocking-in IL-10 in a safe harbor on Chr. 13 using RCL-BC-1 as the parental line by cassette exchange of GFP with IL-10; RCL-BC-1-MIF, an iPSC line generated by knocking-out both HLA Class I (B2M) and Class II (CIITA) genes, and knocking-in MIF in a safe harbor on Chr. 13 using RCL-BC-1 as the parental line by cassette exchange of GFP with MIF; RCL-BC-1-HLAG, an iPSC line generated by knocking-out both HLA Class I (B2M) and Class II (CIITA) genes, and knocking-in HLA-G in a safe harbor on Chr. 13 using RCL-BC-1 as the parental line by cassette exchange of GFP with HLA-G; RCL-BC-1-ILMIF, an iPSC line generated by knocking-out both HLA Class I (B2M) and Class II (CIITA) genes, and knocking-in both IL-10 and MIF (co-expressing via IRES) in a safe harbor on Chr. 13 using RCL-BC-1 as the parental line by cassette exchange of GFP with IL-10 and MIF; and RCL-BC-1-ILMIFHLG, an iPSC line generated by knocking-out both HLA Class I (B2M) and Class II (CIITA) genes, and knocking-in IL-10, MIF and HLA-G (co-expressing via IRES) in a safe harbor on Chr. 13 using RCL-BC-1 as the parental line by cassette exchange of GFP with IL-10, MIF and HLA-G.

Hypoimmunogenic cell lines of the present invention were shown to differentiate into retinal cells, cells of CNS lineages and MSCs. Thus, the modified cells lines of the present invention maintain the same capability as unmodified parent lines in their ability to differentiate into ectoderm, endoderm and mesoderm.

The behavior of these cell lines can be assessed in several well-established in vitro assays. For example, testing of the cell line in a mixed lymphocyte reaction can be used to assess a lack of activation of the innate immune pathway. Mixed peripheral blood cells can be used as well to assess other arms of the immune pathway and interaction between the innate and adaptive pathways. Comparison can be made between the unmodified parental line (control) and the modified lines (hypoimmunogenic lines).

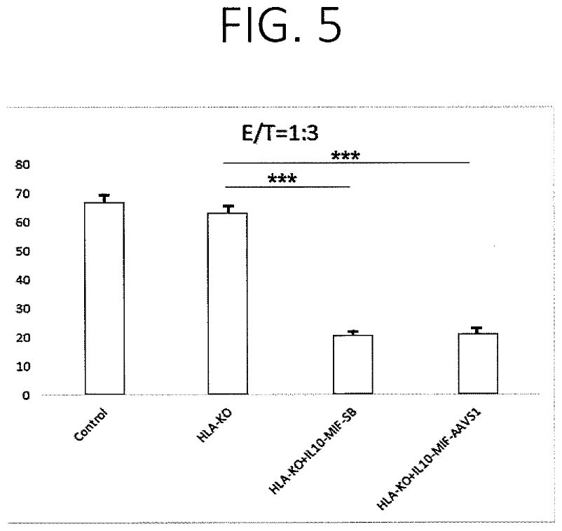

For example, an in vitro immunogenicity assay indicative of NK cell killing measured by LDH toxicity showed the hypoimmunogenic cells lines of the present invention overexpressing IL-10 and/or MIF to elicit less cell killing. See FIG. 5.

The HLA-KO iPSCs were expected to be vulnerable to NK-cell mediated lysis, but not the HLA-KO cells expressing IL-10 and/or MIF. To test this, a NK cell degranulation assay was performed. CD56+NK cells were analyzed by flow cytometry for surface expression of the degranulation marker CD107a as a readout of NK cell activation. As expected, the percentage of CD107a+NK cells in cocultures with HLA-KO cells expressing IL-10 and/or MIF was significantly lower than in HLA-KO cocultures, suggesting that NK cell activity is indeed inhibited by IL-10 and/or MIF. Data showed that NK cell cytotoxicity and degranulation are significantly reduced in HLA-KO lines expressing MIF or IL-10 or both.

In addition, an in vivo immunogenicity assay of xeno-transplantation of hypoimmunogenic cells or neural progenitor cells (NPC) derived from the hypoimmunogenic line of the present invention showed increased survival of the cells in immunocompetent mouse spinal cord compared to controls, as well as decreased expression of IBA1 (Allograft inflammatory factor 1). A combination of HLA-KO with overexpressing MIF and/or IL10 led to increased survival of the cells in the mouse spinal cord compared to cells without the overexpression of IL-10/MIF. More specifically, the HLA-KO cells expressing MIF showed superior ability to survive and differentiate. At 1-week post graft, cells were able to survive and continued to differentiate to form neural rosettes in the mouse spinal cord. At 2-week post graft, despite an increased number of microglia clustered nearby, the grafts persisted along neural lineage differentiation and formed prominent neural rosettes. These results showed IL-10 and MIF did not have a proinflammatory effect as we predicted that these reagents in this configuration were not proinflammatory.

In vivo teratoma assays in normal mice and immunocompromised mice can also be performed.

The following nonlimiting examples are provided to further illustrate the present invention.

### EXAMPLES

### Example 1: Nucleic Acid Construct for IL-10

Details of a nonlimiting example of a construct for insertion/integration of an IL-10 gene are provided below:

Lox2272-CAG-hIL-10 (codon optimized)-SV40pA-Lox511
(SEQ ID NO: 1)
ataacttcgtataggatactttatacgaagttatatttaaatgacattgattattgacta
gttattaatagtaatcaattacggggtcattagttcatagcccatatatggagttccgcg
ttacataacttacggtaaatggcccgcctggctgaccgcccaacgacccccgcccattga
cgtcaataatgacgtatgttcccatagtaacgccaatagggactttccattgacgtcaat
gggtggagtatttacggtaaactgcccacttggcagtacatcaagtgtatcatatgccaa
gtacgccccctattgacgtcaatgacggtaaatggcccgcctggcattatgcccagtaca
tgaccttatgggactttcctacttggcagtacatctacgtattagtcatcgctattacca
tggtcgaggtgagccccacgttctgcttcactctccccatctcccccccctccccacccc
caattttgtatttatttattttttaattattttgtgcagcgatgggggcggggggggggg
gcggcggcagccaatcagagcggcgcgctccgaaagtttccttttatggcgaggcggcgg
ccccgtgccccgctccgcgccgcctcgcgccgcccgccccggctctgactgaccgcgtta
taatgacggctcgtttcttttctgtggctgcgtgaaagccttaaagggctccgggagggc
cgcgtgcggcccgcgctgcccggcggetgtgagcgctgcgggcgcggcgcggggctttgt
gcgctccgcgtgtgcgcgaggggagcgcggccgggggcggtgccccgcggtgcggggggg
cgggggaggggcgcggcggcccccggagcgccggcggctgtcgaggcgcggcgagccgca
gccattgccttttatggtaatcgtgcgagagggcgcagggacttcctttgtcccaaatct
gtgcggagccgaaatctgggaggcgccgccgcaccccctctagcgggcgcggggcgaagc
ggtgcggcgccggcaggaaggaaatgggcggggagggccttcgtgcgtcgccgcgccgcc
accatgttcatgccttcttctttttcctacaggtttaccatgcatagctctgccctgctg
tgctgtctggtcctcttgacaggagtgagggcttcacctggacaaggaacccagtctgaa
aacagctgcactcactttcccggtaatctccccaatatgctgcgggatttgagggacgcc
tttagtagggtgaagacgttctttcagatgaaagaccaactcgataacttgctcctgaaa
gagtcactcttggaggattttaagggttatcttggttgccaggcattgagtgagatgatt
cagttctatttggaagaagtaatgcctcaggctgagaaccaggatcccgacattaaagcg
cacgtaaattccctcggtgaaaatttgaagacactcaggctccggctgcgacgctgccac
cgctttcttccgtgcgaaaataagtctaaagccgtggagcaggtgaagaatgctttcaat
aagctccaagagaaaggcatatataaagcaatgtcagagtttgacattttcatcaactac
atcgaagcctacatgaccatgaaaatcagaaattgaaaacgatcataatcagccatacca
catttgtagaggttttacttgctttaaaaaacctcccacacctccccctgaacctgaaac
ataaaatgaatgcaattgttgttgttaacttgtttattgcagcttataatggttacaaat
aaagcaatagcatcacaaatttcacaaataaagcatttttttcactgcattctagttgtg
gtttgtccaaactcatcaatgtatcttaaaacaaatataacttcgtataatgtatactat
acgaagttat

### Example 2: Nucleic Acid Construct for NIF

A nonlimiting example of inserting construct/sites for overexpressing MIF gene driven by CAG promoter is provided below:

Lox2272-CAG-hMIF-SV40pA-Lox511
(SEQ ID NO: 2)
ataacttcgtataggatactttatacgaagttatatttaaatgacattgattattgacta
gttattaatagtaatcaattacggggtcattagttcatagcccatatatggagttccgcg
ttacataacttacggtaaatggcccgcctggctgaccgcccaacgacccccgcccattga
cgtcaataatgacgtatgttcccatagtaacgccaatagggactttccattgacgtcaat
gggtggagtatttacggtaaactgcccacttggcagtacatcaagtgtatcatatgccaa
gtacgccccctattgacgtcaatgacggtaaatggcccgcctggcattatgcccagtaca
tgaccttatgggactttcctacttggcagtacatctacgtattagtcatcgctattacca
tggtcgaggtgagccccacgttctgcttcactctccccatctcccccccctccccacccc
caattttgtatttatttattttttaattattttgtgcagcgatgggggcggggggggggg
gggcgcgcgccaggcggggcggggcggggcgaggggcggggcggggcgaggcggagaggt
gcggcggcagccaatcagagcggcgcgctccgaaagtttccttttatggcgaggcggcgg
cggcggcggccctataaaaagcgaagcgcgcggcgggcgggagtcgctgcgttgccttcg
ccccgtgccccgctccgcgccgcctcgcgccgcccgccccggctctgactgaccgcgtta
ctcccacaggtgagcgggcgggacggcccttctcctccgggctgtaattagcgcttggtt
taatgacggctcgtttcttttctgtggctgcgtgaaagccttaaagggctccgggagggc
cctttgtgcgggggggagcggctcggggggtgcgtgcgtgtgtgtgtgcgtggggagcgc
cgcgtgcggcccgcgctqcccggcggctgtgagcgctgcgggcgcggcgcggggctttgt
gcgctccgcgtgtgcgcgaggggagcgcggccgggggcggtgccccgcggtgcggggggg
ctgcgaggggaacaaaggctgcgtgcggggtgtgtgcgtgggggggtgagcagggggtgt
gggcgcggcggtcgggctgtaacccccccctgcacccccctccccgagttgctgagcacg
gcccggcttcgggtgcggggctccgtgcggggcgtqqcqcqqqqctcqccgtgccgggcg
gggggtggcggcaggtgggggtgccgggcggggcggggccgcctcgggccggggagggct
cgggggaggggcqcggcggcccccggagcgccggcggctgtcgaggcgcggcgagccgca
gccattgccttttatggtaatcgtgcgagagggcgcagggacttcctttgtcccaaatct
gtgcggagccgaaatctgggaggcgccgccgcaccccctctagcgggcgcggggcgaagc
ggtgcggcqccggcaggaaggaaatgggcggggagggccttcgtgcgtcgccgcgccgcc
qtccccttctccctctccagcctcggggctgtccgcggggggacggctgccttcgggggg
gacggggcagggcggggttcggcttctggcgtqtgaccggcggctctagagcctctgcta
accatgttcatgccttcttctttttcctacaggtttaccatgccgatgttcatcgtaaac
accaacgtgccccgcgcctccgtgccggacgggttcctctccgagctcacccagcagctg
gegcaggccaccggcaagcccccccagtacatcgcggtgcacgtggtcccggaccagctc
atggccttcggcggctccagcgagccgtgcgcgctctgcagcctgcacagcatcggcaag
atcggcggcgcgcagaaccqctcctacagcaagctgctgtgcggcctgctggccgagcgc
ctgcgcatcagcccggacagggtctacatcaactattacgacatgaacgcggccaatgtg
ggctggaacaactccaccttcgcctaaaaacgatcataatcagccataccacatttgtag
aggttttacttgctttaaaaaacctcccacacctccccctgaacctgaaacataaaatga
atgcaattgttgttgttaacttgtttattgcagcttataatggttacaaataaagcaata
gcatcacaaatttcacaaataaagcatttttttcactgcattctagttgtggtttgtcca
aactcatcaatgtatcttaaaacaaatataacttcgtataatgtatactatacgaagtta
t

### Example 3: Cell Differentiation

### Retinal Differentiation

Undifferentiated iPSC on Matrigel-coated plates were treated with retinal induction media containing 2 μM of IWR1 (Sigma Aldrich), 10 μM of SB431542 (Stemgent), 100 nM of LDN193189 (Stemgent) and 10 ng/ml of human recombinant IGF1 (R&D Systems) for 5-7 days with daily medium change. Cells were then dissociated and passaged onto Matrigel-coated plates at a passaging ratio of 1:3 in Neural Stem Cell (NSC) medium that was comprised of DMEM/F-12 1:1 (HyClone), 0.5% Fetal Bovine Serum (FBS, Atlanta Biologicals), 1% Penicillin Streptomycin Amphotericin B (Lonza), 1% Sodium Pyruvate (Corning), 1% Sodium Bicarbonate (Corning), 1% HEPES Buffer (Corning), 1% MEM Non-essential Amino Acids (Corning) and 1% of N1 media supplement (Sigma Aldrich). The neuro-retinal stem cells were serially passaged using Accutase (Global Cell Solutions) at 1:3 ratio upon confluency. For RPE differentiation and maturation, cells at 2 weeks following induction were cultured in RPE medium that contained MEM/EBSS (HyClone) with 1% FBS, 1% Penicillin Streptomycin Amphotericin B, 1% Glutamax (Gibco), 0.25 mg/ml Taurine (Sigma Aldrich), 10 μg/ml Hydrocortisone (Sigma Aldrich) and 0.0065 μg/ml Triiodo-Thyronine (Sigma Aldrich) and 1% N1 media supplement indefinitely till the time of analysis.

### CNS Differentiation

Generation of NSC from iPSC was as previously described (Swistowski et al., 2009). Briefly, confluent iPSC were detached via collagenase and cultured in suspension as EBs in STEMPRO SFM medium (Life Tech.) supplemented with 100 nM LDN193189 (Stemgent), 10 μM SB431542 (Tocris), 2 μM Purmorphamine (Stemgent), 3 μM CHIR99021 (Stemgent), 100 ng/ml Sonic hedgehog (Peprotech) and 100 ng/ml FGF8 (Peprotech) for 8 days. EBs were directed towards neural lineages by the addition of FGF2 and allowed to attach in adherent cultures in NSC maintenance medium (XCell Science Inc.). After attachment, neural tube-like rosette structures were manually dissected and expanded in NSC maintenance medium.

### Example 4: Assessment of Hypoimmunogenicity

Hypoimmunogenicity of cell lines produced in accordance with the present invention can be assessed in a mixed lymphocyte reaction to determine the lack of activation of the innate immune pathway. Mixed peripheral blood cells can be used as well to assess other arms of the immune pathway and interaction between the innate and adaptive pathways. Comparison can be made between the unmodified parental line (control) and the modified lines (hypoimmunogenic lines)

### Example 5: In Vivo Teratoma Assays in Normal and Immunocompromised Mice

Three groups of 5 mice each are tested in normal mice including
Group 1: NCL2-GFP (control, parental line)
Group 2: RCL-B-1: A clone of B2M knock-out (Class I KO) using NCL2-GFP as the parental line)
Group 3: RCL-BC-1: A clone of B2M and CIITA KO (Class I and Class II double KO) using NCL2-GFP as the parental line)
   and in immunocompromised NCr mice including
Group 4: NCL2-GFP (control, parental line)
Group 5: RCL-B-1: A clone of B2M knock-out (Class I KO) using NCL2-GFP as the parental line) and
Group 6: RCL-BC-1: A clone of B2M and CIITA KO (Class I and Class II double KO) using NCL2-GFP as the parental line).

Teratoma size, histology of teratomas and T cell infiltration will be assessed in all groups of animals.

### Example 6: In Vitro NK Call Killing Assay

For NK cell killing assay, hypoimmunogenic cells with and without MIF and IL-10 expression as well as controls were used as target cells. 40K target cells and NK cells at the indicated effector/target ratio were co-incubated in 200 μl NK cell medium in 96-well U bottom for 20 hrs before the supernatants were harvested. After co-incubation, supernatants were collected and analyzed by Pierce™ LDH Cytotoxicity Assay Kit (ThermoFisher Scientific) following the manufacturer's instructions. NK cell medium (RPMI-10) was used as background control. NK cells cultured alone or target cells cultured alone were used as controls for spontaneous LDH release. Lysed target cells at endpoint were used as maximum LDH release.

### Example 7: NR Cell Degranulation Assay

Hypoimmunogenic and unmodified control cells were seeded in 24-well plates 24 hours before the assay. The next day, cells were washed once with PBS before co-incubation with 100K NK cells in NK cell media supplemented with a-CD107a APC (Biolegend) and eBioscience™ Protein Transport Inhibitor Cocktail (ThermoFisher Scientific). After NK cells were added into the wells, the plate was spun down at 2,000 rpm for 5 minutes to achieve sufficient effector-target contact. After a 20 hour-incubation the NK cells were stained with α-CD56 PE (Biolegend) before analysis on a FACSCalibur™ for CD107a cell surface expression. NK cell cultures without target cells were used as negative control. NK cells treated with Cell Activation Cocktail (without Brefeldin A), which includes PMA (phorbol 12-myristate-13-acetate) and ionomycin, were used as positive control for degranulation.

### Example 8: In Vivo Immunogenicity Assay

Hypoimmunogenic and control (unmodified) iPSC lines or neural progenitor cells derived from hypoimmunogenic iPSC lines as wells as from control lines were transplanted into wild-type C57/BL6 adult mouse spinal cord. Animals were euthanized and spinal cords sectioned at 1, 2, and 4 weeks post graft respectively. A few cells from the control were able to survive in the host spinal cord while a lot more HLA-KO and HLA-KO expressing MIF and IL-10 (double knockout of B2M and CIITA) were able to integrate and migrate after 1 week of transplantation. Importantly IBA1 staining showed significantly reduced macrophage/microglia activation in animals transplanted with HLA-KO expressing MIF. These data indicate that HLA-KO expressing IL-10 and MIF enhances immune evasion of the hypoimmunogenic cells in animal host. This also confirms our prediction that MIF and IL-10 did not have a proinflammatory effect.

1: A hypoimmunogenic cell line which does not express Class I epitopes or Class II epitopes and which overexpresses interleukin-10 (IL-10) factor or migration inhibitory factor (MIF) factor.

2: The hypoimmunogenic cell line of claim 1 which does not express Class I epitopes and does not express Class II epitopes.

3: The hypoimmunogenic cell line of claim 1 which overexpresses IL-10 factor and MIF factor.

4: The hypoimmunogenic cell line of claim 1 which is an iPSC or an immortalized cell line.

5: The hypoimmunogenic cell line of claim 1 wherein the cell is any proliferating cell which undergoes sufficient cell divisions to allow insertion and selection of cells which exhibit Class I or Class II epitope elimination and/or an increase in expression of IL-10 factor or MIF factor.

6: The hypoimmunogenic cell line of claim 1 wherein the cells are induced pluripotent stem cells, mesenchymal stem cells, neural stem cells or hematopoietic stem cells.

7: The hypoimmunogenic cell of claim 1 produced by knocked in safe harbor sites or transfection by transposon.

8: A hypoimmunogenic cell line in which expression of IL-10 factor or MIF factor or both is increased and an additional factor which mimics the loss of Class I and/or Class II activity is added.

9: The hypoimmunogenic cell of claim 8 wherein the additional factor is an antisense oligonucleotide or siRNA or modifications for translation that reduces expression of HLA antigens on a surface of the cell or a factor that blocks T and B cell function or alters an inflammatory response.

10: A method for production of a cell line which does not activate innate or adaptive immune responses, said method comprising modifying a cell line to:
not express Class I epitopes or Class II epitopes or to include an additional factor which mimics the loss of Class I and/or Class II activity; and
to overexpress IL-10 factor or MIF factor.

11: The method of claim 10 wherein the cell line is modified to not express Class I epitopes and Class II epitopes.

12: The method of claim 10 wherein the cell line is modified to overexpress IL-10 factor and MIF factor.

13: The method of claim 10 wherein the cell line is modified by knocked in safe harbor sites or transfection by transposon.

14: A nucleic acid construct for insertion or integration into a cell, said construct comprising an IL-10 gene and/or an MIF gene for overexpression in a safe harbor of the cell.


SEQUENCE LISTING (8 sequences)

SEQ ID NO: 1 - DNA, 2530 bp, Artificial sequence
  Synthetic
ataacttcgtataggatactttatacgaagttatatttaaatgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgttgccttcgccccgtgccccgctccgcgccgcctcgcgccgcccgccccggctctgactgaccgcgttactcccacaggtgagcgggcgggacggcccttctcctccgggctgtaattagcgcttggtttaatgacggctcgtttcttttctgtggctgcgtgaaagccttaaagggctccgggagggccctttgtgcgggggggagcggctcggggggtgcgtgcgtgtgtgtgtgcgtggggagcgccgcgtgcggcccgcgctgcccggcggctgtgagcgctgcgggcgcggcgcggggctttgtgcgctccgcgtgtgcgcgaggggagcgcggccgggggcggtgccccgcggtgcgggggggctgcgaggggaacaaaggctgcgtgcggggtgtgtgcgtgggggggtgagcagggggtgtgggcgcggcggtcgggctgtaacccccccctgcacccccctccccgagttgctgagcacggcccggcttcgggtgcggggctccgtgcggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacaggtttaccatgcatagctctgccctgctgtgctgtctggtcctcttgacaggagtgagggcttcacctggacaaggaacccagtctgaaaacagctgcactcactttcccggtaatctccccaatatgctgcgggatttgagggacgcctttagtagggtgaagacgttctttcagatgaaagaccaactcgataacttgctcctgaaagagtcactcttggaggattttaagggttatcttggttgccaggcattgagtgagatgattcagttctatttggaagaagtaatgcctcaggctgagaaccaggatcccgacattaaagcgcacgtaaattccctcggtgaaaatttgaagacactcaggctccggctgcgacgctgccaccgctttcttccgtgcgaaaataagtctaaagccgtggagcaggtgaagaatgctttcaataagctccaagagaaaggcatatataaagcaatgtcagagtttgacattttcatcaactacatcgaagcctacatgaccatgaaaatcagaaattgaaaacgatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttaaaacaaatataacttcgtataatgtatactatacgaagttat

SEQ ID NO: 2 - DNA, 2341 bp, Artificial sequence
  Synthetic
ataacttcgtataggatactttatacgaagttatatttaaatgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgttgccttcgccccgtgccccgctccgcgccgcctcgcgccgcccgccccggctctgactgaccgcgttactcccacaggtgagcgggcgggacggcccttctcctccgggctgtaattagcgcttggtttaatgacggctcgtttcttttctgtggctgcgtgaaagccttaaagggctccgggagggccctttgtgcgggggggagcggctcggggggtgcgtgcgtgtgtgtgtgcgtggggagcgccgcgtgcggcccgcgctgcccggcggctgtgagcgctgcgggcgcggcgcggggctttgtgcgctccgcgtgtgcgcgaggggagcgcggccgggggcggtgccccgcggtgcgggggggctgcgaggggaacaaaggctgcgtgcggggtgtgtgcgtgggggggtgagcagggggtgtgggcgcggcggtcgggctgtaacccccccctgcacccccctccccgagttgctgagcacggcccggcttcgggtgcggggctccgtgcggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacaggtttaccatgccgatgttcatcgtaaacaccaacgtgccccgcgcctccgtgccggacgggttcctctccgagctcacccagcagctggcgcaggccaccggcaagcccccccagtacatcgcggtgcacgtggtcccggaccagctcatggccttcggcggctccagcgagccgtgcgcgctctgcagcctgcacagcatcggcaagatcggcggcgcgcagaaccgctcctacagcaagctgctgtgcggcctgctggccgagcgcctgcgcatcagcccggacagggtctacatcaactattacgacatgaacgcggccaatgtgggctggaacaactccaccttcgcctaaaaacgatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttaaaacaaatataacttcgtataatgtatactatacgaagttat

SEQ ID NO: 3 - RNA, 20 nt, Artificial sequence
  Synthetic
ggccgagaugucucgcuccg

SEQ ID NO: 4 - RNA, 20 nt, Artificial sequence
  Synthetic
acucacgcuggauagccucc

SEQ ID NO: 5 - RNA, 20 nt, Artificial sequence
  Synthetic
cggagcgagagagcacagcg

SEQ ID NO: 6 - DNA, 22 bp, Artificial sequence
  Synthetic
acagcaaactcacccagtctag

SEQ ID NO: 7 - DNA, 21 bp, Artificial sequence
  Synthetic
ccagtctaagggaagcagagc

SEQ ID NO: 8 - DNA, 20 bp, Artificial sequence
  Synthetic
aaactcacccagtctagtgc